(19) United States
(12) Patent Application Publication
(10) Pub. No.: US 2025/0255478 A1
(43) Pub. Date: Aug. 14, 2025

(54) STRABISMUS AND HETEROPHORIA TRAINING SYSTEM AND METHOD USING VR

(71) Applicant: ICU CORPORATION INC., Changwon-si (KR)

(72) Inventor: Dong Hyuk KIM, Changwon-si (KR)

(21) Appl. No.: 19/194,520

(22) Filed: Apr. 30, 2025

Related U.S. Application Data
(63) Continuation of application No. PCT/KR2023/017030, filed on Oct. 30, 2023.

(30) Foreign Application Priority Data
Oct. 31, 2022 (KR) 10-2022-0142879

(51) Int. Cl. A61B 3/08; A61B 3/00; G02B 27/01; G06T 17/00

(52) U.S. Cl. CPC A61B 3/085; A61B 3/0058; G02B 27/017; G06T 17/00

(57) ABSTRACT

The present invention relates to a strabismus and heterophoria training system and method using VR, the system comprising: a VR HMD unit formed to be wearable on both eyes of a user and reproducing a visual training VR video through a display unit located in front of the both eyes; and a manager terminal unit for monitoring the user's gaze in conjunction with the VR HMD and supporting reproduction of the visual training VR video, wherein the manager terminal unit includes: a VR video module for providing a visual training VR video according to the degree of the user's strabismus and heterophoria; and a control unit for controlling image output of the VR video module.

### CROSS REFERENCE TO RELATED APPLICATION

This is a continuation of International Application No. PCT/KR2023/017030 filed on Oct. 30, 2023, which claims priority from Korean Patent Application No. 10-2022-0142879 filed on Oct. 31, 2022. The contents of these applications are incorporated herein by reference in their entireties.

### TECHNICAL FIELD

The present disclosure relates to a strabismus and heterophoria training system and method using virtual reality (VR).

### BACKGROUND

Among visual impairments, strabismus is a disorder where the eyes do not properly aligned, looking at points different from each other. In the case of the strabismus, when one eye looks straight ahead, the other eye is turned inward or outward, or upward or downward, causing deviation.

More precisely, strabismus occurs when the two eyes are not aligned toward the same object. If one eye turns outward relative to the other, the condition is called exotropia. If it turns inward, it is called esotropia.

In addition, heterophoria is a condition of one's eyes with a latent misalignment, wherein when one eye is covered with a cover while looking at an object with both eyes, one's eyes become misaligned, and when the cover is removed, the object is seen properly again.

Unlike strabismus, heterophoria is a condition in which when a patient generally looks at a point with both eyes, eye fatigue occurs in the course of maintaining the lines of sight. When one of the both eyes is covered, a state of the covered eye that turns outward is called exophoria and that turns inward is called esophoria. The cases of hyperphoria and hypophoria may be expressed differently depending on an eye used as reference.

In general, conventional treatment methods for strabismus and heterophoria include a surgery method or a method of using glasses or a prism as a non-surgical method.

The conventional strabismus treatment method using the prism corrects strabismus by way of shifting a prism up and down in front of a strabismus patient's eyes while a therapist holds the prism with one hand. However, such a method is not only a difficult treatment process because the therapist should hold the prism while the treatment is being conducted for a long time, but also causes a lot of inconvenience in the treatment because the face of the patient should be kept in a fixed state for a long time without being able to move freely.

In addition, the deviation degree of strabismus varies greatly depending on the patient's levels of concentration and fatigue. Therefore, although the deviation angle of strabismus changes depending on the patient's condition, the conventional strabismus treatment is not dynamic, so it is difficult to respond to the changing deviation angle of strabismus. In addition, there is a problem in that most of devices used in the conventional strabismus treatment are bulky and patients have limited movement during treatments.

### BRIEF SUMMARY

An objective of the present disclosure is to provide a strabismus and heterophoria training system and method using VR, wherein a position of an image is shifted upward, downward, leftward, and rightward as much as a required amount of prism by using a VR HMD configured to output respective images for left and right eyes, and convergence and divergence exercises are performed by using output images, so as to conduct training for stimulating the extraocular muscles of the eyes.

In addition, another objective of the present disclosure is to provide a strabismus and heterophoria training system and method using VR, wherein training is conducted by expanding the field of view in order to relieve emotional stifling caused by the narrowing field of view that occurs when a screen is shifted during the training.

In addition, a yet another objective of the present disclosure is to provide a strabismus and heterophoria training system and method using VR, wherein training is conducted in a fun and enjoyable way by watching a training VR video provided as VR content.

The technical problems to be solved in the present disclosure are not limited to the technical problems mentioned above, and other technical problems that are not mentioned will be clearly understood by those skilled in the art to which the present disclosure belongs from the following description.

According to the present disclosure for achieving the objectives, there is provided a strabismus and heterophoria training system using VR, the system including: a VR HMD unit provided in a form wearable on both eyes of a user and for playing a visual training VR video through display units that are positioned in front of the both eyes; and an administrator terminal unit for interworking with the VR HMD to monitor the user's eyes and supporting the playing of the visual training VR video, wherein the administrator terminal unit includes: a VR video module for providing the visual training VR video according to degrees of strabismus and heterophoria of users; and a control unit for controlling image output of the VR video module, and the visual training VR video may be played by repeatedly separating and fusing images.

Specifically, the administrator terminal may further include a diagnostic unit for determining the degrees of the strabismus and heterophoria as a failure value of image fusion through the user's eyes.

In addition, the VR video module may include: an image storage unit for storing the images; a field-of-view range storage unit for temporarily storing image range information exposed to the user's field of view; and an image processing unit for generating the visual training VR video exposed according to the degrees of the strabismus and heterophoria of the users by collating the images stored in the image storage unit and the image range information according to the user's field of view.

In addition, according to the degrees of strabismus and/or heterophoria of the users, the image processing unit may fuse and separate images of left and right eyes inward so as to cause the images overlap inward in cases of exotropia and exophoria where the images of the left and right eyes intersect and appear to overlap left and right, fuse and separate images of the left and right eyes outward so as to cause the images overlap outward in cases of esotropia and esophoria where the images of the left and right eyes do not intersect and appear to overlap left and right, fuse and separate images of any one eye downward so as to cause the images to overlap in a case of hypertropia where the images of an strabismus eye are positioned downward and appear to overlap up and down, and fuse and separate images of any one eye upward so as to cause the images to overlap in a case of hypotropia where the images of an strabismus eye are positioned upward and appear to overlap up and down, thereby generating the visual training VR video.

In addition, in the visual training VR video, fusion and separation may performed by the images that are shifted according to the user's prism diopter.

According to the present disclosure for achieving the objectives, there is provided a strabismus and heterophoria training method using VR, the method including: wearing a Virtual Reality Head Mounted Display (VR HMD) so as to cause display units to face both eyes of a user; playing a visual training VR video through the VR HMD according to degrees of strabismus and heterophoria of users; and conducting training by enabling the user to watch the visual training VR video being played, wherein the visual training VR video may perform the training by repeatedly separating and fusing output images and stimulating extraocular muscles.

In addition, the method may further include measuring the degrees of the strabismus and heterophoria as a failure value of image fusion of the images output to left and right eyes of the VR HMD.

In addition, according to the degrees of the strabismus and/or heterophoria of the users, the visual training VR video may fuse and separate images of the left and right eyes inward so as to cause the images overlap inward in cases of exotropia and exophoria where the images of the left and right eyes intersect and appear to overlap left and right, fuse and separate images of the left and right eyes outward so as to cause the images overlap outward in cases of esotropia and esophoria where the images of the left and right eyes do not intersect and appear to overlap left and right, fuse and separate images of any one eye downward so as to cause the images to overlap in a case of hypertropia where the images of an strabismus eye are positioned downward and appear to overlap up and down, and fuse and separate images of any one eye upward so as to cause the images to overlap in a case of hypotropia where the images of an strabismus eye are positioned upward and appear to overlap up and down.

The present disclosure not only has an advantage capable of obtaining the same effect as that of inserting a prism into an eye by outputting an image obtainable with a prism moved according to the degrees and classifications of strabismus and heterophoria of users, but also has an effect of conducting rehabilitation vision training (i.e., divergence/convergence exercises) just by watching images for the training.

In addition, the present disclosure has another effect of expanding a range of the field of view that can be seen by a user with both eyes, thereby relieving emotional stifling of the user watching a visual training VR video.

In addition, the present disclosure has a yet another effect of providing a visual training VR video produced as VR content, thereby enabling a user to receive visual training in an interesting manner.

### BRIEF DESCRIPTION OF DRAWINGS

FIG. 1 illustrates a configurational view illustrating a strabismus and heterophoria training system using VR according to an exemplary embodiment of the present disclosure.

FIG. 2 illustrates an example reference image.

FIG. 3A illustrates an example image illustrating the field of view of a left eye of a normal person when watching the image in FIG. 2 with a VR HMD unit, FIG. 3B is an image illustrating the field of view of a right eye of the normal person, and FIG. 3C is an image illustrating perceived views of the normal eyes.

FIG. 4A illustrates an example image illustrating the field of view of a left eye having esotropia when watching the image in FIG. 2 with a VR HMD unit, FIG. 4B is an image illustrating the field of view of a right eye having esotropia, and FIG. 4C is an image illustrating perceived views of the eyes having esotropia.

FIG. 5 is an example image showing an after-treatment-view of the eye with esotropia, perceiving as in FIG. 4.

FIGS. 6A to 6D illustrate views of prescriptions for fusion of images according to types of strabismus according to the exemplary embodiment of the present disclosure.

FIG. 7 illustrates a strabismus and heterophoria training method using VR according to an exemplary embodiment of the present disclosure.

FIGS. 8A to 8C illustrate example screens for left and right eyes when normal eyes watch a reference screen.

FIGS. 9A to 9C illustrate example views of a prescription for an eye having exotropia in the strabismus and heterophoria training method using VR according to the exemplary embodiment of the present disclosure.

FIGS. 10A to 10C illustrate example views of a prescription for both eyes having exotropia in the strabismus and heterophoria training method using VR according to the exemplary embodiment of the present disclosure.

FIGS. 11A to 11C illustrate example views of a prescription for an eye having esotropia in the strabismus and heterophoria training method using VR according to the exemplary embodiment of the present disclosure.

FIGS. 12A to 12C illustrate example views of a prescription for both eyes having esotropia in the strabismus and heterophoria training method using VR according to the exemplary embodiment of the present disclosure.

FIGS. 13A to 13C illustrate example views of a prescription for hypertropia in the strabismus and heterophoria training method using VR according to the exemplary embodiment of the present disclosure.

FIGS. 14A to 14C illustrate example views of a prescription for hypotropia in the strabismus and heterophoria training method using VR according to the exemplary embodiment of the present disclosure.

Hereinafter, exemplary embodiment of the present disclosure will be described in detail with reference to the accompanying drawings.

In addition, the size or shape of the components shown in the drawings may be exaggerated for clarity and convenience of description, terms specifically defined in consideration of the components and operation of the present disclosure will vary depending on the intention or custom of a user or operator, and it should be understood that definitions of these terms should be made on the basis of the content throughout the disclosure.

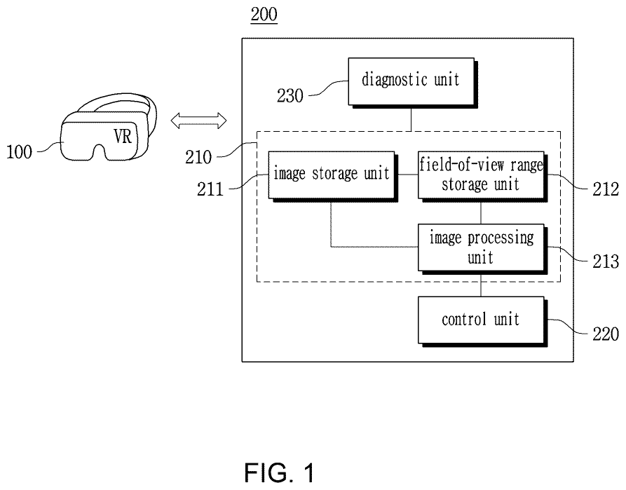

FIG. 1 is a configurational diagram illustrating a strabismus and heterophoria training system using VR according to an exemplary embodiment of the present disclosure. The strabismus and heterophoria training system is composed of a VR HMD unit 100 and an administrator terminal unit 200.

The VR HMD unit 100 is a component formed in a shape mountable on a head and wearable on both eyes of a user, and is provided with two display units positioned in front of both eyes, and is configured to play a visual training VR video through the two display units.

Here, the visual training VR video refers to an image or video for the repetitive separation and fusion of output images, and is a video played to each of the left and right eyes or a video with a position-adjusted screen.

More specifically, in cases of users with strabismus and heterophoria, even when a user wears the VR HMD unit 100, a video or images in the video appear to be two overlapping images, so training is conducted by using images that are provided to the user and output to each of the left and right eyes.

As in the exemplary embodiment of the present disclosure, a screen is shifted leftward, rightward, upward, and downward as much as a required amount of prism to play the video through the VR HMD unit 100, so that the same effect as that of putting a prism in a patient's eye is obtainable, thereby allowing convergence and divergence exercises to be performed without equipment required for vision training.

The administrator terminal 200 interworks with the VR HMD unit 100 to monitor a user's eyes, and processes and controls a visual training VR video.

The administrator terminal 200 includes a VR video module 210, a control unit 220, and a diagnostic unit 230. The VR video module 210, the control unit 220, and the diagnostic unit 230 module may be implemented with a software-controlled microprocessor, a discrete logic circuit, an analog circuit, a digital circuit, a programmed logic device, a memory device containing executing instructions, logic gates, a combination of gates, and/or other circuit components.

The VR video module 210 provides a visual training VR video to the VR HMD unit 100 according to the degrees of strabismus and heterophoria of users. In this case, the VR video module 210 includes an image storage unit 211, a field-of-view range storage unit 212, and an image processing unit 213. The field-of-view range storage unit 212, and the image processing unit 213 may be implemented with a software-controlled microprocessor, a discrete logic circuit, an analog circuit, a digital circuit, a programmed logic device, a memory device containing executing instructions, logic gates, a combination of gates, and/or other circuit components.

The image storage unit 211 may include a memory to store an image or video in the form of VR content. At this time, the image or video is an image to the extent that is visible to a user having a normal eye and is without being influenced by the degree of strabismus of users.

The field-of-view range storage unit 212 temporarily stores image range information that is exposed to a user's field of view.

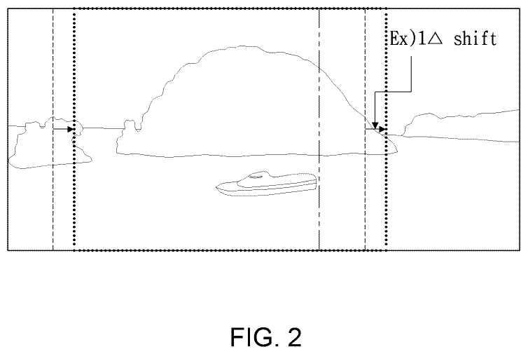

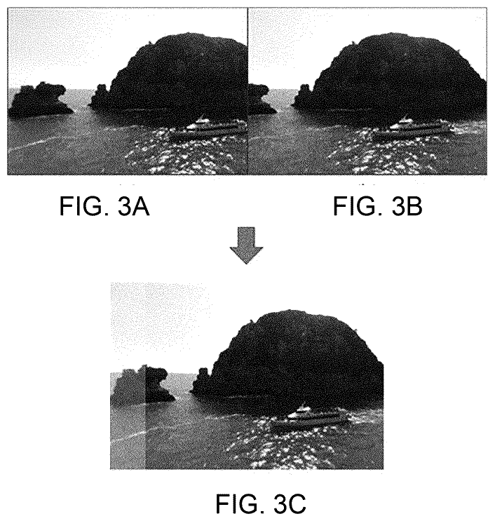

For example, when the image such as that in FIG. 2 is viewed, in a case of normal eyes, respective areas visible to the left and right eyes have a difference in images due to a distance between the two eyes as shown in FIG. 3A and FIG. 3B. However, the areas may be perceived as one image as shown in FIG. 3C.

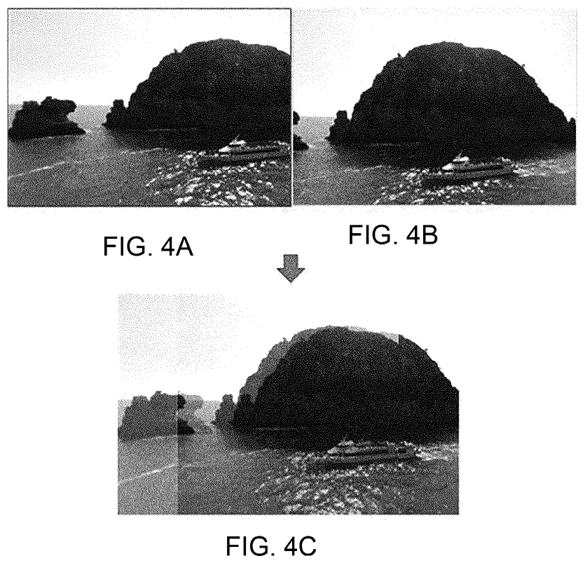

In contrast, in the case of esotropia, as shown in FIGS. 4A and 4B, a difference in the field of view that is farther than a distance between both eyes is generated in areas visible to the left and right eyes, and as shown in FIG. 4C, the images are not perceived as one image, but rather appear to overlap.

In order to resolve this problem, in the exemplary embodiment of the present disclosure, an image in one eye side is shifted so that images do not appear to overlap as much as a difference but appear as one image.

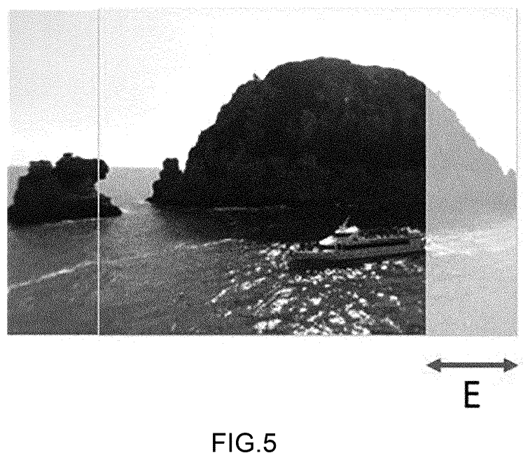

In this case, as a somewhat narrower area than an initially visible area may be visible, the initially visible area is stored so as to be visible, that is, the initial field of view is stored in order to expand a visible area. As shown in FIG. 5, this is for outputting an image by expanding an area E to the extent of a range of the initial field of view when image fuse is performed.

With respect to the expanding of the field of view during strabismus training, for each patient with severe or more severe strabismus, when receiving treatment through VR training, in a case where an image is shifted as much as a prism high relative to one eye, an image of the eye that has shifted within VR tends to be visible while being limited to 30 to 40%. In this case, a patient may complain of discomfort or dizziness during the training, so it is important to expand the field of view to the extent of the image that should originally be visible. Such parts of processing have limitations with optical methods, so the field of view is expanded by processing the images with software.

The image processing unit 213 collects an image stored in the image storage unit 211 as well as image range information based on the field of view that is of each user and stored in the field-of-view range storage unit 212, and generates a visual training VR video that is exposed according to the degrees of strabismus and heterophoria of users

The vision training VR video is generated differently depending on the types of strabismus and heterophoria, and depending on the degrees of strabismus and heterophoria of users.

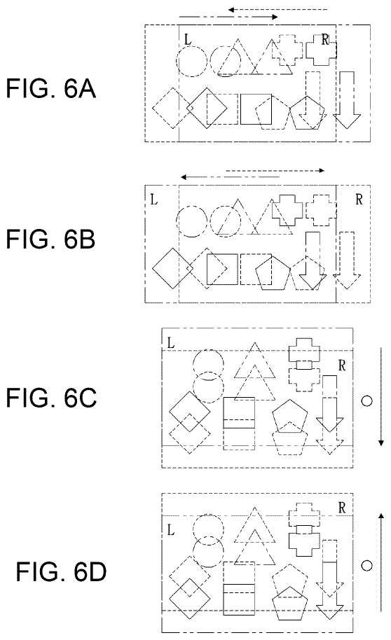

As illustrated in FIGS. 6A to 6D, the methods for image fusion are different from each other depending on the types of strabismus and heterophoria. As illustrated in FIG. 6A, in the cases of exotropia and exophoria in which images of left and right eyes intersect and overlap left and right, “BASE IN” is performed so that the images of the left and right eyes are fused inwardly, thereby causing the images to overlap inward. As illustrated in FIG. 6A, in the cases of esotropia and esophoria in which images of left and right eyes do not intersect and overlap left and right, “BASE OUT” is performed so that the images of the left and right eyes are fused outwardly, thereby causing the images to overlap outward.

For example, for corrective prescriptions, in the cases of patients with exotropia and exophoria, since a right eye is turned to the right and an image of the right eye is turned to the left, an eyeball should be able to be moved inward and the image should be able to be turned to the right.

That is, in the exotropia and exophoria, an eyeball is deviated outward, and in the esotropia and esophoria, an eyeball is deviated inward, but in terms of movement directions of image, in the exotropia, eyeballs are moved crosswise (i.e., a right eye moves to the left and a left eye moves to the right), and in the esotropia, eyeballs are moved ipsilaterally (i.e., a right eye moves to the right and a left eye moves to the left).

In the case of hypertropia in which an image of an eye with strabismus is positioned downward and images overlap top and bottom as illustrated in FIG. 6C, “BASE DOWN” is performed so that images of any one eye is fused downward, thereby causing the images to overlap, and in the case of hypotropia in which an image of an eye with strabismus is positioned upward and images overlap top and bottom as illustrated in FIG. 6D, “BASE UP” is performed so that images of any one eye is fused upward, thereby causing the images to overlap, and thus, fusion is performed in a visual training VR video.

In this case, in the visual training VR video, the images are shifted according to a user's prism diopter, and the images are fused and separated.

Here, a prism diopter is a unit indicating a degree to which light is refracted.

The control unit 220 controls the output or playback of a visual training VR video generated in the image processing unit of the VR video module.

The diagnostic unit 230 determines the degrees of strabismus and heterophoria through eyes of each user and a failure value of image fusion. In the case of strabismus patients, fusion usually fails as soon as a VR HMD unit 100 is put on. A point in time when the images are first fused may be determined as a strabismus value by utilizing a function of shifting images transmitted to the left and right eyes upward, downward, leftward, and rightward and then fusing the images.

More specifically, images separated on both sides appear to overlap in two or appear to be completely separated from each other. The images of the left and right eyes may be horizontally adjusted so that the images are fused into one through calibration. A shifted distance is a strabismus value according to a viewing distance, and may be used for determining the degrees of strabismus and heterophoria.

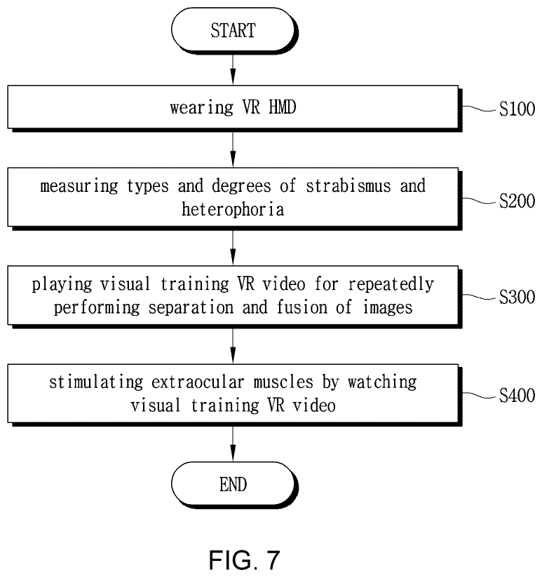

As illustrated in FIG. 7, in a strabismus and heterophoria training method using VR according to the exemplary embodiment of the present disclosure, first, in step S100, a user wears a Virtual Reality Head Mounted Display (VR HMD) so as to allow display units to face both eyes of the user.

Next, in step S200, the types and degrees of strabismus and heterophoria are measured on the basis of a failure value of image fusion of images output to the left and right eye sides of the VR HMD. The present disclosure provides the strabismus and heterophoria training method, and previously measured information for the degrees of strabismus and heterophoria may also be utilized.

Next, in step S300, a visual training VR video is played through the VR HMD according to the degrees of strabismus and heterophoria of users. In this case, the visual training VR video being played is a VR video or an image for repeatedly performing the separation 710 and fusion 720 of output images.

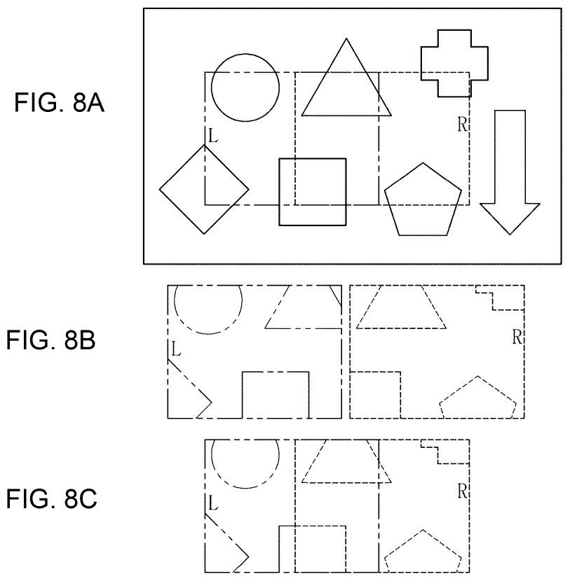

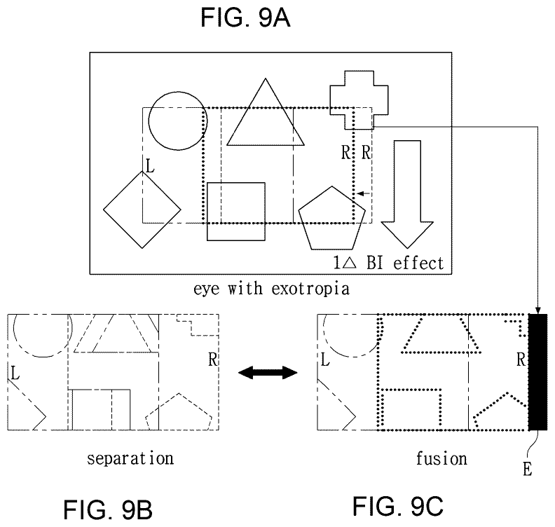

FIG. 8A to 8C are example screens for left and right eyes when normal eyes watch a reference screen. FIG. 8A illustrates the reference screen for describing FIGS. 9 to 15 afterward. FIG. 8B illustrates respective screens which are for the left and right eyes and shown by the normal eyes. FIG. 8C illustrates each screen that is of the left and right eyes and is actually shown. The views of the screens varies depending on the types and degrees of strabismus and heterophoria, so this will be used for description.

FIGS. 9A to 14C illustrate respective images of prescriptions for each case according to the types and degrees of strabismus and heterophoria. In FIGS. 9A to 14C, a dashed-double dotted line indicates a screen of a normal left eye, a dashed line indicates a screen of a normal right eye, a dashed-single dotted line indicates a screen of a prism-prescribed left eye, and a dotted line indicates a screen of a prism-prescribed right eye.

That is, the present exemplary embodiment of the present disclosure is characterized by repeatedly training a separation screen in which screens are separated and a fusion screen in which the screens are fused.

In the case of exotropia, images seen from the left and right eyes intersect and overlap left and right. As illustrated in FIG. 9A, in the cases of exotropia or exophoria in any one eye (an eye), when glasses are prescribed, a prism “BASE IN” is prescribed to show an image of the right eye that is shifted as much as a required amount of prism toward the nose so that the image overlaps inward, thereby achieving the same effect as prism glasses and conducting training by repeatedly performing the process of separating as in FIG. 9B and fusing as in FIG. 9C.

In particular, in the present exemplary embodiment of the present disclosure, in order to prevent the field of view from being narrowed during fusion. The fusion is performed by including an extended section E that maintains the field of view that is seen during separation. That is, the prism-prescribed screen of the right eye expands further as much as a difference from the screen for the right eye.

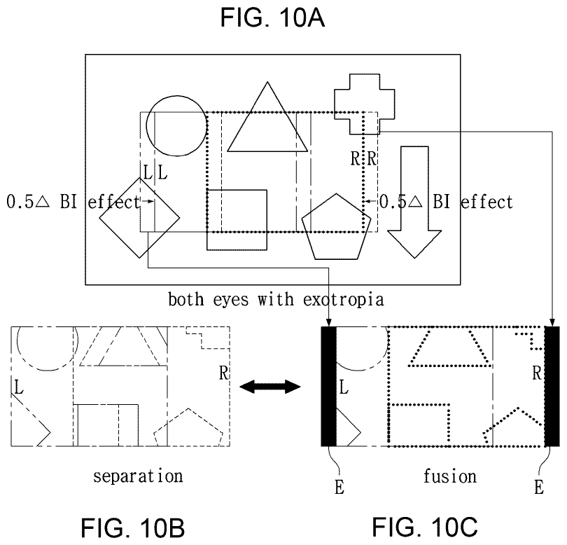

In addition, as illustrated in FIGS. 10A to 10C, in the cases of exotropia and exophoria in both eyes, “BASE IN” is performed inward on the both eyes so that images overlap, and a screen is shown in which the both eyes' images are shifted as much as a required amount of prism toward the nose, whereby training is conducted by repeatedly performing the process of separating as in FIG. 10B) and fusing as in FIG. 10B.

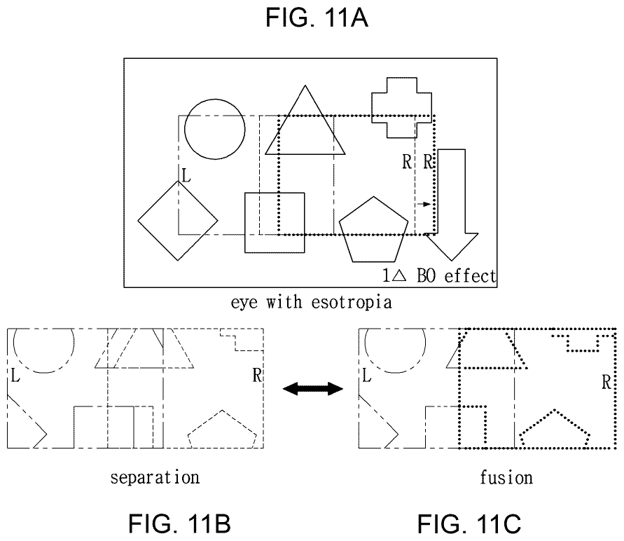

As illustrated in FIG. 11A, in the cases of esotropia and esophoria in any one eye (an eye), images do not intersect but overlap left and right, so prism “BASE OUT” is prescribed when glasses are prescribed. In a visual training VR video, a screen is shown in which the right eye's image is shifted as much as a required amount of prism toward an ear, thereby achieving the same effect as prism glasses and conducting training by repeatedly performing the process of separating as in FIG. 11B and fusing as in FIG. 11C.

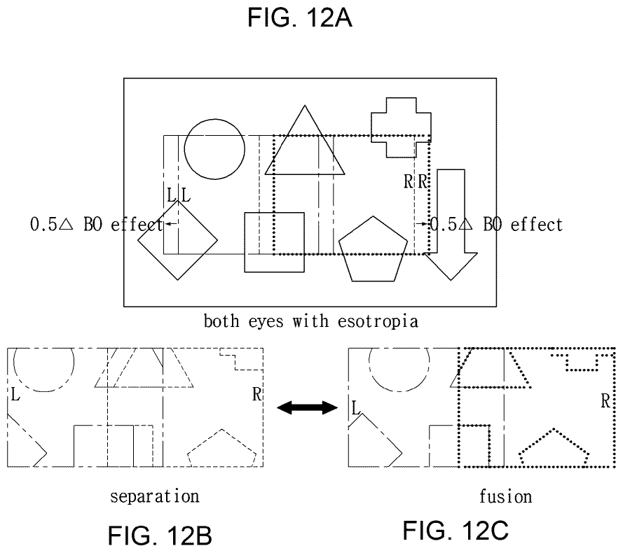

In addition, as illustrated in FIG. 12A, in the cases of esotropia and esophoria in both eyes, “BASE OUT” is performed outward on the both eyes so that the images overlap, and a screen is shown in which the both eyes' images are shifted as much as a required amount of prism toward the ears, whereby training is conducted by repeatedly performing the process of separating as in FIG. 12B and fusing as in FIG. 12C.

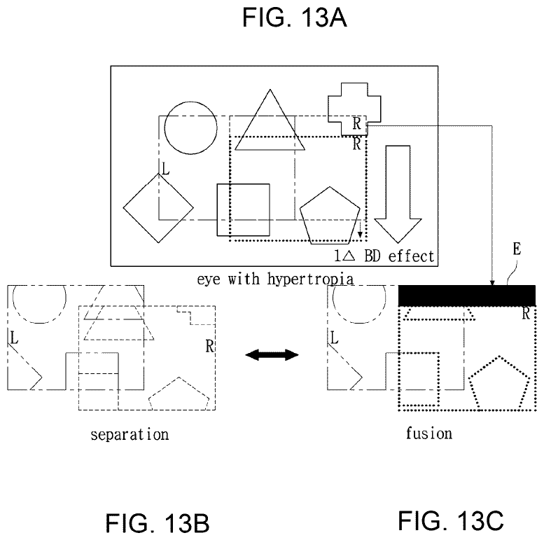

As illustrated in FIG. 13A, in the cases of hypertropia and hyperphoria in which images seen from the left and right eyes overlap up and down, prism “BASE DOWN” is prescribed when glasses are prescribed, so as to shift the images upward. In the visual training VR video of the exemplary embodiment of the present disclosure, the images should be shifted upward, so a fixed screen is shown in which the images are shifted downward as much as a required amount of prism from an existing image, thereby having the same effect as a prism glasses prescription and conducting training by repeatedly performing the process of separating as in FIG. 13B and fusing as in FIG. 13C.

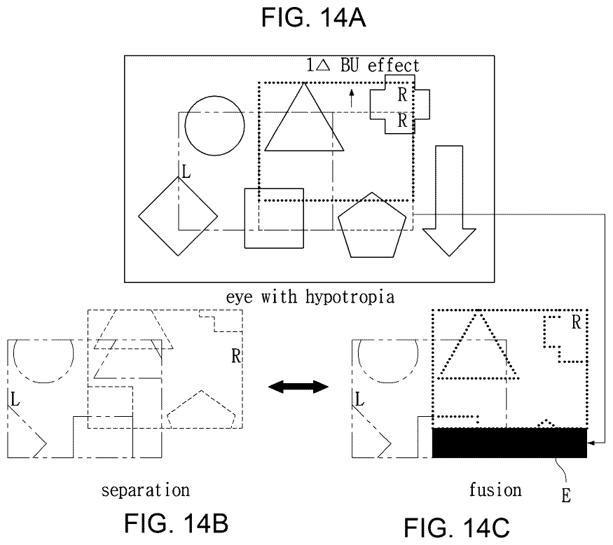

As illustrated in FIG. 14A, in the cases of hypotropia and hypophoria in which images seen from the left and right eyes overlap upward, prism “BASE UP” is prescribed when glasses are prescribed, so as to shift the image downward. In the visual training VR video of the exemplary embodiment of the present disclosure, the images should be shifted downward, so a fixed screen is shown in which the images are shifted upward as much as a required amount of prism from an existing image, thereby having the same effect as a prism glasses prescription, and conducting training by repeatedly performing the process of separating as in FIG. 14B and fusing as in FIG. 14C.

Lastly, in step S400, the user watches the visual training VR video currently playing so as to perform training with the movement of eyeballs according to the movement of the images, thereby stimulating extraocular muscles. The extraocular muscles are the muscles that move the eyeballs.

Therefore, the present disclosure has not only an advantage capable of obtaining the same effect as inserting a prism into an eye by a prism outputting a shifted image according to the degrees and classifications of strabismus and heterophoria of users, but also an effect of conducting rehabilitation vision training (i.e., divergence/convergence exercises) just by watching the training images.

In addition, the present disclosure has another effect of expanding a range of the field of view that can be seen by a user with both eyes, thereby relieving emotional stifling of the user watching a visual training VR video.

The present disclosure has a yet another effect of providing a visual training VR video produced as VR content, thereby enabling a user to receive visual training in an interesting manner.

Although the exemplary embodiments according to the present disclosure have been described above, these are merely exemplary, and those skilled in the art will understand that various modifications and equivalent embodiments are possible. Therefore, the true technical protection scope of the present disclosure should be defined by the following patent claims.

### INDUSTRIAL APPLICABILITY

The present disclosure relates to a strabismus and heterophoria training system and method using VR.

The present disclosure has not only an advantage capable of obtaining the same effect as inserting a prism into an eye by a prism outputting a shifted image according to the degrees and classifications of strabismus and heterophoria of users, but also an effect of conducting rehabilitation vision training (i.e., divergence/convergence exercises) just by watching the training images.

1. A strabismus and heterophoria training system using virtual reality (VR), the system comprising:
a VR HMD unit provided in a form wearable on both eyes of a user and for playing a visual training VR video through display units that are positioned in front of the both eyes; and
an administrator terminal unit for interworking with the VR HMD to monitor the user's eyes and supporting the playing of the visual training VR video,
wherein the administrator terminal unit comprises:
a VR video module for providing the visual training VR video according to degrees of strabismus and heterophoria of users; and
a control unit for controlling image output of the VR video module, and
the visual training VR video is played by repeatedly separating and fusing images.

2. The system of claim 1, wherein the administrator terminal further comprises:
a diagnostic unit for determining the degrees of the strabismus and heterophoria as a failure value of image fusion through the user's eyes.

3. The system of claim 1, wherein the VR video module comprises:
an image storage unit for storing the images;
a field-of-view range storage unit for temporarily storing image range information exposed to the user's field of view; and
an image processing unit for generating the visual training VR video exposed according to the degrees of the strabismus and heterophoria of the users by collating the images stored in the image storage unit and the image range information according to the user's field of view.

4. The system of claim 1, wherein according to the degrees of strabismus and/or heterophoria of the users, the image processing unit fuses and separates images of left and right eyes inward so as to cause the images overlap inward in cases of exotropia and exophoria where the images of the left and right eyes intersect and appear to overlap left and right, fuses and separates images of the left and right eyes outward so as to cause the images overlap outward in cases of esotropia and esophoria where the images of the left and right eyes do not intersect and appear to overlap left and right, fuses and separates images of any one eye downward so as to cause the images to overlap in a case of hypertropia where the images of an strabismus eye are positioned downward and appear to overlap up and down, and fuses and separates images of any one eye upward so as to cause the images to overlap in a case of hypotropia where the images of an strabismus eye are positioned upward and appear to overlap up and down, thereby generating the visual training VR video.

5. The system of claim 1, wherein in the visual training VR video, fusion and separation is performed by the images that are shifted according to the user's prism diopter.

6. A strabismus and heterophoria training method using VR, the method comprising:
wearing a Virtual Reality Head Mounted Display (VR HMD) so as to cause display units to face both eyes of a user;
playing a visual training VR video through the VR HMD according to degrees of strabismus and heterophoria of users; and
conducting training by enabling the user to watch the visual training VR video being played,
wherein the visual training VR video performs the training by repeatedly separating and fusing output images and stimulating extraocular muscles.

7. The method of claim 6, further comprising:
measuring the degrees of the strabismus and heterophoria as a failure value of image fusion of the images output to left and right eyes of the VR HMD.

8. The method of claim 6, wherein according to the degrees of the strabismus and/or heterophoria of the users, the visual training VR video fuses and separates images of the left and right eyes inward so as to cause the images overlap inward in cases of exotropia and exophoria where the images of the left and right eyes intersect and appear to overlap left and right, fuses and separates images of the left and right eyes outward so as to cause the images overlap outward in cases of esotropia and esophoria where the images of the left and right eyes do not intersect and appear to overlap left and right, fuses and separates images of any one eye downward so as to cause the images to overlap in a case of hypertropia where the images of an strabismus eye are positioned downward and appear to overlap up and down, and fuses and separates images of any one eye upward so as to cause the images to overlap in a case of hypotropia where the images of an strabismus eye are positioned upward and appear to overlap up and down.
